(19) United States
(12) Patent Application Publication
(10) Pub. No.: US 2016/0067366 A1
(43) Pub. Date: Mar. 10, 2016

(54) Multi-layered incense device and method of making such

(71) Applicant: Heidi Elise Newell, Willowick, OH (US)

(72) Inventor: Heidi Elise Newell, Willowick, OH (US)

(21) Appl. No.: 13/999,998

(22) Filed: Apr. 15, 2014

Related U.S. Application Data
(62) Division of application No. 12/456,035, filed on Jun. 10, 2009, now abandoned.

(51) Int. Cl. A61L 9/02

(52) U.S. Cl. CPC A61L 9/02

(57) ABSTRACT

A multi-layered incense device comprising a rigid support layer in a shape of an ornate design or pattern, at least one water settable wood powder layer disposed on each side of the support layer, an aromatic layer disposed on the wood powder layer, and a powdered dye layer disposed on the aromatic layer. The multi-layered device can be produced by cutting a template to form a support layer, moistening the support layer with water and then coating it with a wood powder mixture. The wood powder mixture is sprinkled with water for setting. The support layer and the wood powder mixture is then placed between two layers of vinyl covered screen for curing, and is then sprinkled with water, an aromatic layer and a powdered dye layer. The support layer and its various mixture and layers are placed between two vinyl covered screens and cured.

### BACKGROUND OF THE INVENTION

I. Field of the Invention

This invention relates generally to a method of manufacturing incense into specific shapes and designs which allows the burn to follow a trail or route predetermined by the embedded template and allows the incense shape/design to burn independent of a heat source. These shapes can be used in religious rituals, festivities, or for other incidents or situations where incense would be appropriate, so that novelty and amusement can be derived from the incense and specific value can be applied to the incense, and thus enhance the presence the incense represents as well as augment the incident or situation where the incense is being used.

II. Description of the Prior Art

Incense has traditionally been manufactured in stick, coil, loose powder and cone form. Once lit, these types of incense burn arbitrarily and simultaneously over the whole incense piece with no predictable pattern. They usually have no visual connotation to the purposes behind their use. For example someone could do a ritual incorporating religious icons placed around them and have a stick of incense for aroma, but the physical appearance of the incense would have no direct connection to the ritual; for the purpose of the ritual, the incense has absolutely no aesthetic value.

This present invention, for the purpose of this example, would be a piece of incense created by using a template to form it's base and coating it with wood powders to create a burnable shape which might comprise a design of religious significance. Therefore, the incense is more durable and the role of the incense has been elevated from merely providing accompanying scent to the ritual to simultaneously complementing the ritual for which it is being used visually. Hence, its aesthetic contribution enhances the ritual experience.

Past incense makers have extruded incense dough into shapes; however, even when the dough is thoroughly dry, the shapes break apart and crumble easily and are therefore not stable enough to be packaged and sold to the general public. This present invention solves that problem by adding a template which forms the base of the incense shape and when coated with the wood powders and dried thoroughly, creates a sturdy, rigid form of incense in a variety of designs that is far more durable and hence suitable for shipping. An added benefit to this present invention is that unlike some other incense shapes made from dough, this present invention, while having the template, still burns without the use of a separate heat source such as charcoal or an electric burner.

### Brief Summary of the Invention

The purpose of the present invention is to, among other things; provide a creative, novel way to incorporate sacred imagery and unique designs together with traditional incense for use in meditation, as a point of concentration and in various religious rites. The images that this incense represent, as described above, are integral for these purposes, as is the presence and use of incense. This invention successfully marries these two crucial elements together for a novel approach to the purposes listed. Another purpose of this present invention is to create a rigid support for incense to be shaped around so the incense shape/design will be more sturdy and durable enough to withstand the rigors of handling and shipping to the general public.

The present invention is a method for embodying an incense-coated template in a variety of ornate and complex designs or patterns by cutting a template of the desired shape or design by hand or machine; sprinkling the shape with water until sufficiently moistened; sprinkle coating the shape with a wood powder mixture; lightly sprinkling the shape with water again to “set” the wood powders; allowing the shape to dry thoroughly; repeating these steps until the coating is at the desired thickness and completely encases the template; coating the shape with a fragrance in various forms; sprinkle coating the shape with a colored powder and then sprinkling the shape with water to set the color; allowing the shape to dry thoroughly.

### BRIEF DESCRIPTION OF THE DRAWINGS

FIG. 1 presents the schematic diagram showing a process of manufacturing an incense-coated template in a symbolic shape.

### DESCRIPTION OF THE PREFERRED EMBODIMENT

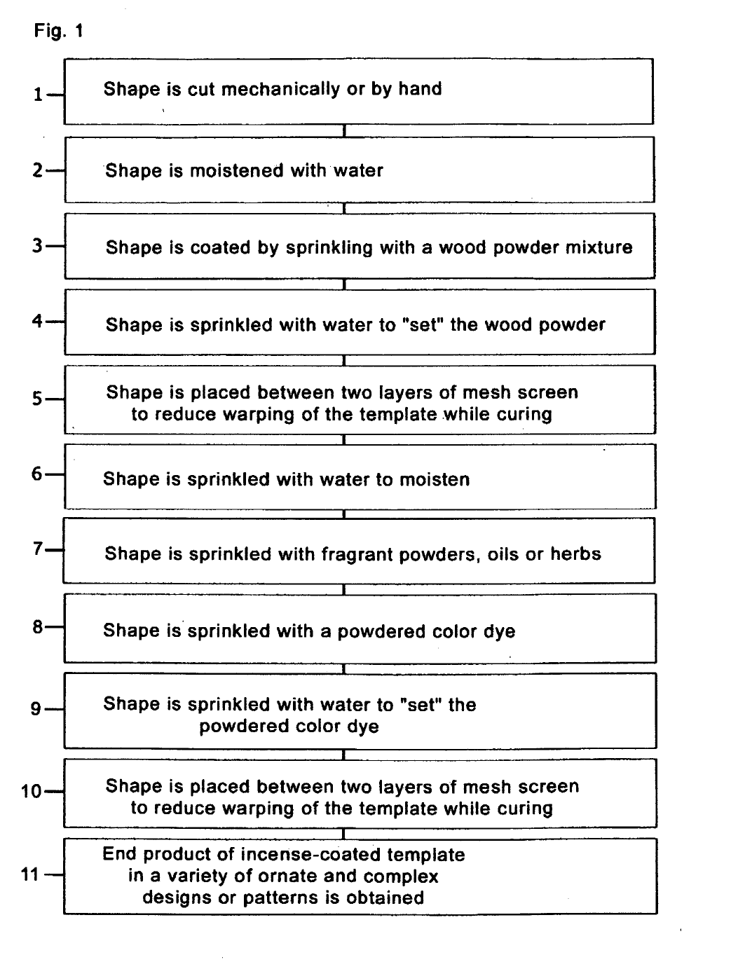

Refer to FIG. 1, the first step of the process of manufacturing an incense-coated template in a variety of ornate designs and patterns is cutting the shape/design out of any variety of options from the group consisting of bamboo paper, rice paper, papyrus paper or thin sheets of wood 1 and is cut mechanically or by hand. The shape/design is lightly sprinkled with water until sufficiently moistened 2. Said shape/design is then sprinkle-coated with a mixture of the group consisting of various wood powders such as Makko, Laha, Dar and any fragrant wood powder 3. For this present invention the following wood powders are mixed together for use: 2 parts Sandalwood wood powder, 2 parts Makko wood powder and 1 part Dar wood powder. The shape/design is lightly sprinkled with water again to “set” the wood powders 4. Next, the shape/design is placed between two layers of vinyl-covered screen 5. Small weights are then placed on the screen at various positions in order to ensure the finished product does not warp during the curing process. After this initial curing, steps 2-5 are repeated on both sides of the shape/design as needed until the finished product attains an average thickness of 2 mm and the template is thoroughly encased in the dried wood powder. Said shape/design is once again lightly sprinkled with water 6 and then sprinkled with an aromatic such as scented oils, herbs, fragrant wood powders or powdered incense 7 on each side of the shape/design. The shape/design is then sprinkled with a powdered dye on each side to attain the desired coloration 8. Said shape/design is sprinkled with water to “set” the powdered color dye 9. The shape/design is placed again between the two layers of vinyl-covered screen to cure 10 and once thoroughly dry an incense-coated template in a variety of ornate and complex designs or patterns is obtained 11.

Even though this invention has been described by employing the process stated in the preferred embodiment, it is understood that an array of materials, variations, substitutions and modifications can be made without departing from the scope and spirit of the present invention.

What is claimed is:

1. A multi-layered incense device comprising:
a rigid support layer in a shape of an ornate design or pattern,
at least one water settable wood powder layer disposed on each side of the support layer,
an aromatic layer disposed on the wood powder layer, and
a powdered dye layer disposed on the aromatic layer.

2. The multi-layered incense device as claimed in claim 1, in which the rigid support layer is in the shape of a symbol of religious significance.

3. The multi-layered incense device as claimed in claim 1, in which the support layer is made from any one of the following materials:
bamboo paper,
rice paper,
papyrus paper or
thin sheets of wood.

4. The multi-layered incense device as claimed in claim 1, in which the wood powder layer is made using any one of the following materials:
Makko,
Laha, or
Dar.

5. The multi-layered incense device as claimed in claim 1, in which the wood powder layer is made using a mixture of any two or all of the following materials: Makko, Laha, or Dar.

6. The multi-layered incense device as claimed in claim 5, in which the ratio of the two materials used for the wood powder layer is 3 parts Makko wood powder and 1 part Dar wood powder.

7. The multi-layered incense device as claimed in claim 1, in which the wood powder layer is made from 2 parts Sandalwood wood powder, 2 parts Makko wood powder and 1 part Dar wood powder.

8. The multi-layered incense device as claimed in claim 1, in which the wood powder layer comprises a material having adhesive properties for binding additional substances to an external surface of the incense device.

9. The multi-layered incense device as claimed in claim 1, in which the wood powder layer includes a fragrant wood powder.

10. The multi-layered incense device of claim 1, in which the wood powder, when dried thoroughly, increases the rigidity of the device.

11. The multi-layered incense device of claim 1, in which the rigid support layer is completely encased by the wood powder, when assembled.

12. The multi-layered incense device as claimed in claim 1, in which the aromatic layer is made from any one of the following materials:
scented oils,
herbs,
fragrant wood powders or
powdered incense.

13. The multi-layered incense device of claim 1, in which the aromatic layer, when dried thoroughly, increases the rigidity of the device.

14. The multi-layered incense device as claimed in claim 1, which has an overall average thickness of approximately 2 mm.

15. A method of making a multi-layered incense device, the method comprising the steps of:
a. Cutting a template to form a support layer;
b. Moistening the support layer with water;
c. Coating the support layer with a wood powder mixture;
d. Sprinkling the wood powder mixture with water to set the wood powder mixture;
e. Placing the support layer and wood powder between two layers of vinyl covered screen;
f. Curing the support layer and wood powder to dry the wood powder;
g. Sprinkling the wood powder mixture with water;
h. Sprinkling the wood powder mixture with an aromatic layer;
i. Sprinkling the aromatic layer with a powdered dye layer;
j. Placing the support layer, the wood powder mixture, the aromatic layer and the powdered dye layer between two vinyl covered screens; and
k. Curing the support layer, the wood powder mixture, the aromatic layer and the powdered dye layer.

16. The method of making a multi-layered incense device as claimed in claim 15, in which step a) is carried out mechanically.

17. The method of making a multi-layered incense device as claimed in claim 15, in which step a) is carried out manually.

18. The method of making a multi-layered incense device as claimed in claim 15, further comprising the step of placing at least one weight on the vinyl covered screen to reduce warping during subsequent curing step f).

19. A kit of parts for making a multi-layered incense device according to the method as claimed in claim 15, the kit of parts comprising:
a rigid support layer in a shape of an ornate design or pattern,
at least one water settable wood powder layer,
an aromatic layer,
a powdered color dye layer; and
two layers of vinyl covered screen.

20. A kit of parts for making a multi-layered incense device as claimed in claim 19, further comprising a plurality of small weights.
